(19) United States
(12) Patent Application Publication
(10) Pub. No.: US 2015/0165536 A1
(43) Pub. Date: Jun. 18, 2015

(54) METHOD FOR ASSEMBLING, BY BRAZING, A SUBSTRATE COMPRISING PYROCARBON WITH PARTS COMPRISING PYROCARBON

(71) Applicant: COMMISSARIAT A L'ÉNERGIE ATOMIQUE ET AUX ÉNERGIES ALTERNATIVES, Paris (FR)

(72) Inventors: Valérie Chaumat, Saint-Paul De Varces (FR); Philippe Bucci, Engins (FR); Olivier Mailliart, Caen (FR)

(73) Assignee: COMMISSARIAT A L'ENERGIE ATOMIQUE ET AUX ENERGIES ALTERNATIVES, Paris (FR)

(21) Appl. No.: 14/366,172

(22) PCT Filed: Dec. 21, 2012
(86) PCT No.: PCT/EP2012/076751

§ 371 Date: Jul. 30, 2014

(30) Foreign Application Priority Data
Dec. 22, 2011 (FR) 1162338

(51) Int. Cl. B23K 1/00; B23K 35/30; A61L 31/02; B32B 18/00; B32B 3/14; B23K 1/19; C22C 5/06

(52) U.S. Cl. CPC B23K 1/0008; B23K 1/19; B23K 35/30; B23K 35/3006; C22C 5/06; B32B 18/00; B32B 3/14; A61L 31/022; B32B 2250/02; B32B 2255/205; B32B 2535/00; C04B 2237/16; C04B 2237/125; C04B 2237/363

(57) ABSTRACT

A method is provided for assembling by brazing a substrate comprising at least one external pyrocarbon surface with parts such as beads each comprising at least one external pyrocarbon surface. The method includes depositing a brazing material composition consisting of an alloy of one or several metal(s) or metalloid(s) on at least one portion to be assembled of the external pyrocarbon surface of the substrate. The assembly so obtained is also described.

### TECHNICAL FIELD

The present invention relates to a method for assembling by brazing a substrate comprising pyrocarbon with parts comprising pyrocarbon.

More specifically, the invention relates to a method for assembling by brazing a substrate comprising at least one external pyrocarbon surface with parts, also called added elements, each comprising at least one external pyrocarbon surface.

The substrate and the added elements, such as beads, may consist of pyrocarbon, or else they may be formed with a first material or core material, generally biocompatible, covered with a pyrocarbon skin.

The assembling method according to the invention is generally applied at a brazing temperature not exceeding 1,700° C., preferably between about 1000° C. and 1,450° C.

The method according to the invention notably allows the making of implants intended to be introduced into a patient.

The technical field of the invention may therefore be defined as that of implants comprising pyrocarbon.

### STATE OF THE PRIOR ART

Pyrocarbon is biocompatible and is used as an implant or part of an implant for prostheses of bone portions. It is also used as a moveable interposition implant between two bone surfaces and ensures the joint function.

It is particularly of interest for this function since it has a very good friction coefficient with bone and a Young's modulus close to that of the bone, which avoids generating wear and tear of the bone and of the implant.

In the literature, implants of the prosthesis type, for replacing bones or bone portions generally consist of two portions, a male part and a female part. At least one of these parts includes a rod which will engage into a bone portion adjacent to the bone or a bone portion which is desirably replaced, in order to maintain the prosthesis against the bone portion. These operations for attaching the prosthesis are delicate to apply and force the making of a hole in the bone for letting through the rod. These attachments do not leave the possibility to the surgeon of replacing the prosthesis in the case of wear, except generating consequent damages on the bone(s) to which the rod is attached.

Thus, document [1] describes an implant comprising a pyrocarbon head, intended to replace at least one lateral articular portion of a bone receiving transverse loads and a supporting element connected to the head and intended to be attached to said bone.

Therefore there exists a need for implants which may be attached without it being necessary to pierce the bone. The head is translationally moveable relatively to the supporting element in at least one direction, when the implant is in place in the patient, corresponding to a direction of said transverse loads.

Interposition implants between two bone surfaces are notably described in document [2].

These pyrocarbon-based implants, in particular for which at least the external surface is in pyrocarbon, are moveable between the bone surfaces.

This requires a housing to be generated for these implants; and sometimes one or several, more or less worn, bone surfaces facing the moveable implant have to be suitably trimmed for making this housing.

Therefore there exists a need for interposition implants which do not require generation of a housing, by trimming the bone surfaces for receiving them.

The goal of the present invention is to provide a method for preparing a pyrocarbon substrate or for which at least the external surface is in pyrocarbon, notably intended to be used as an implant which inter alia meets the needs mentioned above.

### DESCRIPTION OF THE INVENTION

This goal and further ones are achieved according to the invention with a method for assembling by brazing a substrate comprising at least one external surface made of pyrocarbon with parts each comprising at least one external surface made of pyrocarbon, wherein the following successive steps are carried out:
a brazing material composition consisting of an alloy of one or several metals or metalloids is deposited on at least one portion to be assembled of the external pyrocarbon surface of the substrate;
the parts are deposited on the brazing material composition, so that the brazing material composition, upon melting, is in contact with the portion to be assembled of the external surface of the substrate on the one hand and with a portion to be assembled of the external pyrocarbon surface of each of the parts on the other hand, so that the shape of the parts is not changed and remains apparent, so as to leave free empty spaces between the parts, and so that the parts are not embedded in the brazing material composition;
the assembly formed by the substrate, the brazing material composition and the deposited parts is heated in vacuo or in a neutral gas atmosphere, at a brazing temperature sufficient for totally or at least partially melting the brazing material composition, and less than 1,700° C. ;
the substrate, the brazing material composition and the parts are cooled until solidification of the brazing material composition.

The melting of the brazing material composition is generally considered as being total when it is found in a liquid state, at a temperature greater than or equal to the liquidus.

The melting of the brazing material composition is generally considered as partial when it is found in a state which may be described as semi-solid, viscous, softened, at a temperature located between the solidus and the liquidus.

The parts should not be embedded in a large amount of brazing material composition, they are only assembled through a portion to be assembled, which is generally their base to the substrate.

The global shape of the parts should remain apparent, visible, identifiable in order to ensure a role of surface structuration with free empty spaces between the parts for allowing growth of cells, such as bone cells.

The portions of the parts which are not in the assembling area, in other words the portions of the external pyrocarbon surface of each of the parts, different from the portions to be assembled of the external surface of each of the parts may either be covered with the brazing material composition or not, with the proviso that the thickness of the covering remains very small relatively to the dimensions of the part, in order not to modify the shape of the parts and to leave free empty spaces between the parts.

The portion to be assembled of the external surface of the substrate and the portion to be assembled of the external surface of each of the parts are generally made of pyrocarbon.

Preferably, the totality of the external surface of the substrate and the totality of the external surface of the parts are made of pyrocarbon.

Two or more from among the deposited parts may be in contact with each other or else all the deposited parts may not be in contact with each other.

The method according to the invention is a brazing method.

It is known that brazing is a technique which allows assembly of at least two parts by melting a supplied, added material called brazing material or brazing alloy, the melting temperature of which is less than that of the materials to be assembled.

The material to be assembled according to the invention is pyrocarbon which does not have any melting point but which nevertheless may be degraded by a temperature above 1,700° C.

The substrates, parts, deposits or coatings made of pyrocarbon are generally manufactured at a very high temperature, for example around 1,400° C. and should therefore not be exposed to temperatures above 1,700° C. during the brazing cycle. This is why the brazing temperature applied in the method according to the invention does not exceed 1,700° C. since beyond this temperature the pyrocarbon would be degraded.

Advantageously, the brazing is carried out at a brazing temperature greater by at least 15° C., preferably by at least 30° C. than the melting temperature of the brazing material.

Preferably, the brazing temperature is from 980° C. to 1,450° C.

The method according to the invention, which is a method for assembling by brazing a substrate and specific parts at least the external surface of which is made of a specific material, i.e. pyrocarbon, has never been described or suggested in the prior art.

More generally, the assembling of parts made of a material based on pyrocarbon, by brazing, has never been described or suggested in the prior art.

Thus, document [4] proposes a method for assembling at least two parts made of materials based on silicon carbide by non-reactive refractory brazing in which the brazing material composition consists of 40 to 97 atomic % of silicon and of 60 to 3 atomic % of another element from Cr, Re, V, Ru, Ir, Rh, Pd, Co, Pt, Ce, Zr and in which, before brazing, a SiC and/or C reinforcement is added to the composition.

This document therefore specifically relates to the brazing of parts based on silicon carbide.

Brazing material compositions containing silicon like those described in [4] may be applied in the method according to the invention, but the fact that a brazing material composition is suitable for brazing parts based on silicon carbide does not at all mean that this same brazing material composition may be suitable for brazing pyrocarbon parts.

Indeed, the behavior of brazing material compositions is extremely difficult to predict and is highly random and depends notably on the material constituting the parts to be assembled.

The fact that a brazing material composition is suitable for brazing parts made of a given material and has excellent properties and gives excellent results for brazing parts made of this given material does not by any means mean that this same composition will have the same properties and will give the same results or will even be suitable for brazing parts made of another material.

In particular, it was absolutely impossible with reference to document [4] to predict the proper behavior of the interfaces between the pyrocarbon and the brazing alloys of this document, and in particular the mechanical behavior at these interfaces.

Document [5] describes a method for covering parts made of materials based on silicon carbide by means of a covering composition consisting of a brazing material composition selected from the same compositions as the ones mentioned in document [4], to which is added a SiC and/or C reinforcement.

There again, the covered parts are exclusively parts based on SiC and are not pyrocarbon parts.

Further, the deposits prepared in document [5] cover well and are very homogeneous with the purpose of protecting the SiC-based ceramic.

According to the invention, a homogeneous and protective coating is not formed but rather structuration of the external pyrocarbon surface of an implant is achieved by forming irregularities (asperities) and holes between the brazed added elements, for example, brazed beads, for example at their base.

The method according to the invention notably gives the possibility of preparing implants with a pyrocarbon substrate or coated with pyrocarbon, provided with a structured surface on which the bone cells will be able to develop.

The structured surface according to the invention is constituted by added, applied, parts, elements, such as beads, covered with pyrocarbon and assembled by brazing on the external surface of the substrate. This structured surface is found facing the bone when the implant is positioned in the patient.

The method according to the invention gives the possibility of preparing implants which do not have the drawbacks of the implants of the prior art and which solve the problems of the implants of the prior art.

Thus, as regards the implants of the prosthesis type, the method according to the invention gives the possibility of making a structured surface on the substrate made of pyrocarbon or with a pyrocarbon surface.

This structuration is obtained by assembling by brazing added elements, for example beads, made of pyrocarbon or coated with pyrocarbon, on the substrate.

Thus, it is unnecessary to pierce the bone for attaching the implant. The faulty bone portion is machined and/or slightly polished, the implant is placed on the bone with the structured portion orientated towards the bone. The assembly is maintained without moving during the phase when the bone cells develop in the structured portion until the bone and the implant are secured together. This thus avoids piercing of the bone.

As regards interposition implants, the method according to the invention allows preparation of the structured implants formed with an external pyrocarbon surface on which are assembled by brazing added elements, for example beads, made of pyrocarbon or coated with pyrocarbon.

The bone surfaces facing the implants are simply slightly machined and polished and it is no longer necessary to significantly trim these bone surfaces for making a housing therein.

The implants prepared by the method according to invention with their structured pyrocarbon surface are then placed on the slightly machined and polished surfaces.

The brazing material composition, brazing alloy applied according to the invention should give the possibility of obtaining a brazing plateau at a maximum temperature of 1,700° C. in a secondary vacuum or in a neutral atmosphere.

Next, the brazing alloy should also have limited and controlled reactivity while forming strong bonds with the pyrocarbon.

The inventors have surprisingly shown that brazing alloys known for brazing other materials than pyrocarbon were suitable for brazing pyrocarbon.

These brazing material compositions, brazing alloys, belong to three categories of brazing alloys, brazing material compositions.

Thus, the brazing material composition may be selected from alloys based on an element X comprising more than 40 atomic % of an element X selected from Ni, Fe, Co, Pd, or Pt (first category of brazing alloys).

Let us indeed specify that herein, by “alloy based on one element or several elements”, is generally meant that said alloy comprises more than 40 atomic % of said element or of said elements.

Or else, the brazing material composition may be selected from alloys based on Ag or Ag and Cu containing at least one reactive element such as Ti at a concentration of less than 10 atomic %; and alloys based on Ag containing Si at a concentration of less than 40 atomic % (second category of brazing alloys).

Among commercial brazing alloys belonging to the second category, mention may notably be made of AgCuTi alloys available under the names of Ticusil® and Silver-ABA®.

Or else further, the brazing material composition may be selected from silicon-based alloys containing Si at a concentration of more than 40 atomic % and which have limited and controlled reactivity with pyrocarbon (third category of brazing alloys).

Let us specify that by “alloy having limited reactivity with pyrocarbon”, is generally meant that there is dissolution of the carbon and formation of SiC in an area for which the thickness is less than 20 μm, or even 10 μm, therefore with a generally very small thickness as compared with the thickness of the pyrocarbon layer.

Let us specify that by “alloy having controlled reactivity with pyrocarbon”, is generally meant that the reaction between the alloy and the pyrocarbon is reproducible and substantially identical from one part, such as a bead, to the other.

The preferred brazing alloys in the method according to the invention are brazing alloys which belong to the second and third categories. The brazing alloys which belong to the third category are further preferred.

The brazing material compositions of the third category may be selected from binary alloys consisting of silicon at a concentration of more than 40 atomic % and by an element selected from Cr, Re, V, Ru, Ir, Rh, Pd, Co, Pt, Ce, Zr, Ti, Ag, Au, Cu, Nd, Pr, Y and Hf, i.e. the SiCr, SiRe, SiV, SiRu, SiIr, SiRh, SiPd, SiCo, SiPt, SiCe, SiZr, SiTi, SiAg, SiAu, SiCu, SiNd, SiPr, SiY and SiHf binary alloys.

Preferably, the brazing material compositions belonging to the third category may be selected from binary alloys (A) consisting of silicon and zirconium, binary alloys (B) consisting of silicon and titanium, binary alloys (C) consisting of silicon and cobalt, and binary alloys (D) consisting of silicon and silver.

Advantageously, the binary alloy (A) in atomic percentages consists of 75% to 97% of silicon, and of 25% to 3% of Zr; preferably from 89% to 97% of silicon, and from 11% to 3% of zirconium.

Advantageously, the binary alloy (B) in atomic percentages consists of 60% to 97% of silicon, and of 40% to 3% of Ti; preferably from 80% to 97% of silicon, and from 20% to 3% of titanium.

Advantageously, the binary alloy (C) in atomic percentages consists of 60% to 97% of silicon, and of 40% to 3% of Co; preferably from 75% to 97% of silicon, and from 25% to 3% of cobalt.

Advantageously, the binary alloy (D) in atomic percentages consists of 5% to 97% of silicon, and of 95% to 3% of Ag; preferably from 10% to 90% of silicon, and from 90% to 10% of silver.

Advantageously, the substrate consists of pyrocarbon, or else the substrate consists of a core made of a first material preferably selected from biocompatible materials such as zirconia, graphite or silicon carbide; at least one portion of the external surface of the core made of a first material, and preferably the totality of the external surface of the core made of a first material being coated with a pyrocarbon coating or skin.

Advantageously, the parts consist of pyrocarbon or else each of the parts consist of a core made of a first material preferably selected from biocompatible materials such as zirconia, graphite or silicon carbide; at least one portion of the external surface of the core made of a first material and preferably the totality of the external surface of the core made of a first material being coated with a pyrocarbon coating or skin.

Advantageously, the parts appear as spherical or spheroidal beads, preferably with a grain size comprised between 2.5 mm and a few tens of micrometers, for example between 2.5 mm and 100 μm, preferably between 2.5 mm and 50 μm.

Advantageously, a brazing composition powder may be formed, said powder is suspended in an organic binder so as to obtain a brazing composition suspension, paste or slurry, and said suspension or slurry is deposited on at least one portion to be assembled of the external pyrocarbon surface of the substrate.

Advantageously, said substrate is an implant, or an implant portion, for prostheses of bony portions; or an interposition implant between bone surfaces.

The method according to the invention, notably in the case when the preferred brazing alloys A, B, C, and D are applied, ensures that the added parts or elements such as beads, and the substrate, keep all their integrity at the end of the assembling operation.

The method according to the invention, notably in the case when the preferred brazing alloys A, B, C, and D are applied, give the possibility of obtaining, inter alia, the following properties:
A good wetting of the surface of the added parts, elements such as beads, which is in contact with the substrate, and of the substrate, with the brazing alloys.

This surface of the parts which is in contact with the substrate is generally defined as the base of these parts such as beads.

This thus leads to a good surface for attaching the parts or added elements, such as beads, with for example, an assent of the brazing material onto the edges of the bead and proper filling of the joint between the bead and the substrate, which improves the mechanical behavior while allowing better transfer of the stresses.

However, according to the invention, the added elements, parts, such as beads are not “embedded” into the brazing material (i.e. the solidified brazing material composition after melting) but only attached through one of their surfaces or bases to the substrate in order to generate a structured surface i.e. with raised/recessed, protruding areas irregularities (asperities), recessed areas, and free, empty, spaces between the parts; and/or with strong roughness; which allow the growth of cells such as bone cells between the added parts or elements such as beads.

The global shape of the added elements, parts such as beads, is not modified, remains unchanged, apparent in order to guarantee surface structuration with spaces available for the growth of cells such as bone cells.

Only the “base” of the parts is modified because of the formation of a joint between the part and the substrate.

Indeed, according to the invention, the parts are deposited on the brazing material composition, so that the brazing material composition during its melting is in contact with the external surface of the substrate on the one hand and with a portion to be assembled of the external pyrocarbon surface of each of the parts on the other hand.

The parts, added elements may be in contact with each other, or else they may not be in contact with each other.

When the added elements, parts are not in contact with each other, they may be described as distinct elements, parts.

When the added parts, elements are in contact with each other, anyway there should be spaces between the added elements but this does not prevent there being a few contact points between the added elements.

The preferred embodiment is the one in which the added parts, elements are not in contact with each other.

In every case, there exist free spaces, volumes, a “porosity” between the added parts, elements, in which the growth of cells as bone cells, may take place.

Further, the parts are not totally embedded in a large amount of brazing material composition, they are generally only assembled through the base to the pyrocarbon substrate.

The global shape of the parts should remain apparent in order to ensure the structuration role and thereby allow growth of cells such as bone cells.

The portions of the parts which are not in the assembly area may either be covered or not with brazing material composition, provided that the covering thickness remains very small relatively to the dimensions of the part in order not to modify the shape of the parts. These parts thus form a surface having protrusions, raised/recessed portions, irregularities (asperities), recesses, roughness which is also favorable for the growth of the cells, such as bone cells.

The brazing method leads to:
good adherence, good mechanical attachment, between the base of the added parts, elements, such as the base of the beads, and the substrate after brazing. Strong bonds are formed between the pyrocarbon and the substrate and the parts and the brazing material (i.e. the cooled and solidified brazing material composition).
good mechanical strength of the joints between the added parts, elements, such as beads, the brazing metal and the substrate.
very moderate reactivity of the brazing alloy on the pyrocarbon substrates or with a pyrocarbon skin (see Examples). There are no complex and porous embrittlement areas at the interface. Only a slight dissolution of the carbon of the pyrocarbon into the brazing material has been observed.

Generally, the method according to the invention mainly applies to the making of implants, but other applications of the method according to the invention are for example in the field of high temperature technologies.

The invention further relates to the assembly obtained with the method according to the invention.

### BRIEF DESCRIPTION OF THE DRAWINGS

FIG. 1 is a graph which illustrates the thermal brazing cycle used in Examples 1 and 2.

In abscissas, the duration t is plotted in minutes from beginning of the heat treatment and in ordinates the temperature T is plotted in ° C.

FIG. 2 is a photograph which shows the zirconia disc coated with pyrocarbon no. 2 of Example 2, with zirconia beads coated with pyrocarbon wetted by the molten brazing material composition.

FIG. 3A is a photograph which shows assembled beads on disc no. 2 of Example 2.

FIG. 3B is a photograph which shows assembled beads on disc no. 2 of Example 2.

FIG. 3C is a photograph which shows assembled beads on disc no. 7 of Example 2.

FIG. 4 is a photograph taken with a scanning electron microscope (SEM) of the beads/substrate-disc junction obtained in Example 2.

The scale indicated in FIG. 4 represents 300 μm.

FIG. 5 is a photograph which shows the assembled beads on the disc with a brazing material SiTi in Example 4.

FIG. 6 is a photograph taken with a scanning electron microscope (SEM) which shows the joint between a bead and the substrate obtained in Example 4.

The bead, brazing material and substrate assembly is coated, cut out and polished before observation with the SEM. The scale indicated in FIG. 6 represents 500 μm.

FIG. 7 is a photograph taken with the scanning electron microscope (SEM), which is an enlarged view of a portion of the photograph of FIG. 6, and which shows a slight bead/brazing material and substrate/brazing material reactivity.

The scale indicated in FIG. 7 represents 100 μm.

FIG. 8 is a photograph taken with the scanning electron microscope (SEM) which shows the assembly between two beads and the substrate obtained in Example 7.

The scale indicated in FIG. 8 represents 1 mm.

FIG. 9 is a photograph taken with the scanning electron microscope (SEM), which is an enlarged view of a portion of the photograph of FIG. 8 and which shows the joint between a bead and the substrate.

The scale indicated in FIG. 9 represents 100 μm.

FIG. 10 is a photograph taken with the scanning electron microscope (SEM), which is an enlarged view of another portion of the photograph of FIG. 8 and which shows the joint between a bead and the substrate.

FIG. 11 is a photograph taken with the scanning electron microscope (SEM) which shows the assembly between a bead and the substrate obtained in Example 8.

The bead, brazing material and substrate assembly is cut out and polished, but not coated, before observation with the SEM.

The scale indicated in FIG. 11 represents 1 mm.

FIG. 12 is a photograph taken with the scanning electron microscope (SEM), which is an enlarged view of a portion of the photograph of FIG. 11, which shows the interface between the pyrocarbon and the brazing material and shows slight reactivity (in grey).

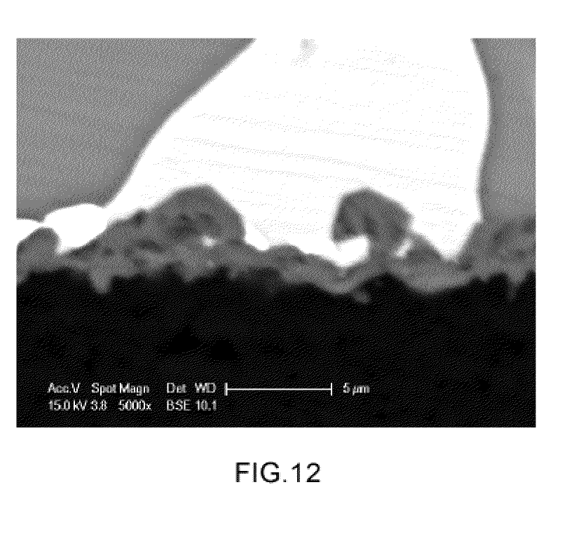

The scale indicated in FIG. 12 represents 5 μm.

### DETAILED DESCRIPTION OF PARTICULAR EMBODIMENTS

The first step of the method according to the invention first of all consists of preparing, elaborating a brazing material composition, in other words a brazing alloy.

The brazing material composition is generally a powdery composition in the form of a powder but it may also appear in the form of sheets.

Two ways for preparing the brazing composition are described hereafter but there exists many other ways for preparing the brazing composition and the two ways for preparing the brazing composition which are more particularly described below are described without this being any limitation.

Thus, according to a first way for preparing the brazing composition, one begins by synthesizing first of all from pure elements, an alloy containing the elements of the brazing material composition in the retained proportions for these elements in the composition.

The pure elements are the elements Si and Zr for the composition, the preferred alloy A according to the invention; the elements Si and Ti for the composition, the preferred alloy B according to the invention; the elements Si and Co for the composition, the preferred alloy C according to the invention; the elements Si and Ag for the composition, the preferred alloy D according to the invention.

The synthesis of such an alloy is for example accomplished by introducing the pure elements for example in the form of pieces or other in the intended proportions for the brazing material composition to be prepared in a refractory crucible, for example, in an alumina crucible.

The assembly formed by the crucible and the elements of the brazing composition placed in the crucible, is positioned in a heating apparatus, such as a graphite oven or a metal oven and is heated to a temperature comprised between a minimum temperature Tmin and a maximum temperature Tmax for a duration from 10 to 60 minutes, in vacuo, typically from 10⁻⁴ to 10⁻⁵ mbars (for example for the synthesis of SiCo) or in a neutral gas atmosphere, for example an argon atmosphere (for example for the synthesis of AgSi).

A neutral gas atmosphere is actually indispensable under certain conditions, for certain brazing materials in order to avoid vaporization of a component of the brazing material, for example in the case of an AgSi brazing composition.

Table 1 below specifies the temperatures Tmin and Tmax for the different brazing alloys, brazing material compositions. Tmin and Tmax therefore define the domain, the range of temperatures for elaborating brazing alloys.

TABLE 1
Temperature range for elaborating brazing alloys.

| | Composition range | | |
|---|---|---|---|
| Alloy | (% at. Si) | Tmin (° C.) | Tmax (° C.) |
| A | 75 to 80 | 1,600° C. | 1,650° C. |
| | 80 to 85 | 1,500° C. | 1,600° C. |
| | 85 to 88 | 1,450° C. | 1,500° C. |
| | 88 to 97 | 1,420° C. | 1,450° C. |
| B | 60 to 75 | 1,450° C. | 1,570° C. |
| | 75 to 97 | 1,380° C. | 1,450° C. |
| C | 60 to 90 | 1,350° C. | 1,400° C. |
| | 90 to 97 | 1,400° C. | 1,450° C. |
| D | 5 to 20 | 1,010° C. | 1,050° C. |
| | 20 to 30 | 1,050° C. | 1,200° C. |
| | 30 to 50 | 1,200° C. | 1,300° C. |
| | 50 to 97 | 1,300° C. | 1,420° C. |

This heating thermal treatment leads to the melting of the elements of the brazing material composition.

After cooling, the homogeneous desired final brazing alloy is obtained as an ingot.

The ingot of the obtained alloy may then be milled in any adequate apparatus, for example in a mortar, in order to obtain a powder with an adequate grain size, i.e. for which the grains for example have a size defined by their largest dimension, such that the diameter in the case of a spherical or spheroidal shape is between 1 and 300 μm.

This powder forms the brazing material composition.

Or else, notably in the case of alloys that are the most concentrated in Ag, the ingot may be transformed into a sheet by rolling for example.

This sheet then constitutes the brazing material composition.

According to a second way for preparing the brazing material composition, the binary brazing alloy used according to the invention, a powder of an element of the brazing material composition and a powder of an intermetallic compound of the elements which constitute the brazing composition, are weighed in the proportions retained for the brazing material composition and these powders are then mixed in a “Turbula” for at least 30 minutes.

Thus, in the case of the preferred brazing alloys A, B and C used according to the invention, weighing is performed by observing the portions retained for the brazing material composition of the Si powder, and respectively for powders of the compound ZrSi₂ for composition A, of TiSi₂ for composition B, and of CoSi₂ for composition C, and then these powders are mixed.

Let us note that for the composition D, there is no defined compound between Ag and Si, and that in this case, the powders of Si and of Ag are therefore simply mixed. It should be emphasized that the handling of the silver powder should be accomplished under a protective atmosphere, such as an argon atmosphere, in order to avoid oxidation of this powder.

The powder consisting of the mixture of the powders of an element of the brazing material composition and of an intermetallic compound of the elements which make up the brazing material composition, for example, the mixture of the Si and the intermetallic compound, ZrSi₂ for composition A, TiSi₂ for composition B, and CoSi₂ for composition C, constitutes the brazing material composition in this second way.

Said intermetallic compounds such as, ZrSi₂, TiSi₂, or CoSi₂ may be synthesized or else this is a commercial compound appearing in the form of an intermetallic compound powder with known grain size and purities.

The pure silicon powder may be prepared from pieces of pure silicon milled in any adequate apparatus, for example, in a mortar, in order to obtain an adequate grain size powder for which the grains for example have a diameter from 1 to 250 μm.

Instead of being prepared in this way, said pure silicon powder may also be a commercial powder with known grain size and purity. From among these commercial powders, mention may for example be made of: pure Si powder, of the CERAC® brand, with a purity of 99.5% or 99.99% and a grain size of the order of 50 μm.

The brazing material composition powder is then conventionally suspended in a binder, cement, liquid organic gel, preferably both viscous and tacky, in order to obtain a slurry suspension of a brazing material composition optionally added with a reinforcement, for example made of SiC, made of C or made of zirconia.

The reinforcement may optionally improve the mechanical behavior of the brazing material.

It should be noted that the spreading of the brazing material is already excellent without any reinforcement.

The binder, cement, gel generally decomposes, for example between 100° C. and 300° C. without leaving any traces.

From among the cements, mention may for example be made of cements available from the corporation Wall Colmonoy® under the name of NICROBRAZ® such as NICROBRAZ® 650 cement.

From among the organic gels, mention may be made of the organic gels available from VITTA®.

The second step of the method according to the invention, generally consists of achieving the assembling by brazing, strictly speaking.

During this step, assembling is accomplished of one substrate on the one hand and of discrete elements, parts on the other hand which may also be described as added elements, parts.

According to the invention, at least one portion of the external surface of the substrate and preferably the totality of the external surface of the substrate, i.e. the surface to be assembled by brazing with the added elements is made of pyrocarbon.

The substrate may consist of pyrocarbon or else the substrate may consist of a core in a first material including at least one portion of the external surface of the core in a first material and preferably the totality of the external surface of the core in a first material, coated with a pyrocarbon coating, skin.

The substrate covered with a structured surface obtained by brazing of the added elements is generally used as an implant or implant portion intended to be introduced into a patient for prostheses of bone portions or still further as a moveable interposition implant between two bone surfaces for ensuring a joint function. Consequently, the substrate and the core have a shape which corresponds to the shape of the implant. This shape notably depends on the intended location for the implant in the body of the patient, because this implant is either a total or partial implant or else is still a moveable interposition implant.

The invention may be applied with the same success regardless of the shape of the substrate and/or of its size.

Because the substrate covered with a structured surface obtained by brazing of the added elements which is generally used as an implant or implant portion intended to be introduced into a patient, the core generally consists of one or several first biocompatible material(s).

As examples of such materials, mention may notably be made of zirconia, graphite, or silicon carbide.

The coating of all or part of the external surface of the core in a first material with a pyrocarbon coating may be accomplished with any method known to the man skilled in the art.

Notably, it is possible to use a method for depositing pyrocarbon in a fluidized bed as the one described in document [3], to the description of which reference may be made.

The thickness of this coating may range from 1 to 1,000 μm, preferably from 100 to 500 μm.

This coating may either be polished or not.

Just like a substrate, the added elements may be formed with pyrocarbon or else each of the added elements may be formed by a core in a first material, at least one portion of the external surface of the core in a first material and preferably the totality of the external surface of the core in a first material coated with a pyrocarbon skin, coating.

The core of these added elements generally consists of one or several first biocompatible material(s).

As examples of such materials, mention may notably be made of zirconia, graphite, or silicon carbide.

The coating of the external surface of the core made of a first material of the elements added with a pyrocarbon coating may be accomplished with any method known to the man skilled in the art.

It is notably possible to use a method for depositing pyrocarbon in a fluidized bed like the one described in document [3], to the description of which reference will be made.

The thickness of this coating may range from 1 to 1,000 μm, preferably from 100 to 500 μm.

This coating may either be polished or not.

The added elements may appear in different shapes, for example in the form of polyhedra, spheres or beads.

Preferably, the added elements appear in the form of spherical or spheroidal beads.

The beads may have a grain size typically comprised between 2.5 mm and a few tens of micrometers, for example between 2.5 mm and 100 μm, preferably between 2.5 mm and 50 μm.

Prior to assembling, the surfaces of the substrate and of the added elements to be assembled are generally degreased, cleaned, in an organic solvent for example of the ketone ester, ether, alcohol type or a mixture thereof.

A preferred solvent is acetone or an acetone/ethyl alcohol/ether mixture for example in the proportions ⅓, ⅓, ⅓; it is also possible to successively clean the surfaces with several different solvents, for example with acetone and then with ethanol. The surfaces are then dried.

The suspension, slurry, paste of the brazing material composition prepared as described earlier, is spread out, coated, applied homogeneously, uniformly, for example with a brush, a spatula, a paintbrush, or by means of a syringe optionally attached to an automated system, or by any other means allowing deposition of a uniform brazing slurry layer, on a portion or on the totality of the external pyrocarbon surface of the substrate such as a substrate with a zirconia core coated with pyrocarbon.

The amount of slurry, suspension, paste of brazing material composition is typically from 10 mg/cm² to 100 mg/cm², preferably from 10 to 50 mg/cm². This amount varies according to the composition of the brazing metal.

The added elements such as beads are then deposited on the surface of the substrate, thus coated beforehand with a slurry, suspension of brazing material composition.

The deposition of the added parts, elements such as beads, may be accomplished for example by means of a spatula or by means of an automated system giving the possibility of depositing these added elements such as beads.

The amount of added parts, elements deposited depends on the size of these added elements.

Thus, in the case when the added elements are beads, the amount of deposited beads depends on the grain size of the beads and may range from 50 mg/cm² to 300 mg/cm², preferably from 100 mg/cm² to 150 mg/cm².

According to the invention, the parts are deposited on the brazing material composition so that the brazing material composition during melting is in contact with the external surface of the substrate on the one hand and with a portion to be assembled of the external pyrocarbon surface of each of the parts, on the other hand.

It is essentially or even exclusively the base which is in contact with the substrate which is in contact with the brazing material composition. In this way, the parts are not “embedded”, coated in the brazing material composition once the latter has solidified and the parts are assembled only through one of their surfaces such as their base with the substrate.

Let us note that the global shape of the parts should be retained so that they may play their role of structuration of the substrate.

The portions of the parts which are not in the assembly area may be covered with brazing material composition or not, provided that the thickness of the covering is thin, remains very small relatively to the dimensions of the parts in order to retain their global shape and free empty spaces for notably bone growth.

Thus, it is considered that the deposition layer illustrated in FIG. 6 is not an embedded bead since its global shape is again found.

Further, the added elements, parts may possibly have points of contact with each other, with possible formation of a joint through these contact points.

These contact points are generally discrete and with a small surface area as compared with the external surface of the parts and of the empty free spaces which should always exist between the parts.

Next, the parts ready to be brazed, i.e. the substrate provided with a layer of brazing material composition slurry on which are deposited the added elements, are placed in a heating device such as a brazing oven or subject to heating by any other adequate means.

The oven is preferably a graphite oven and the brazing operations may be carried out in vacuo or in an atmosphere of neutral gas, but the oven may also be a metal oven and the brazing operations may similarly be carried out in vacuo or in a neutral gas atmosphere.

Generally, the vacuum is a secondary vacuum, i.e. the pressure is from 10⁻³ Pa to 10⁻⁵ Pa, for example, 10⁻⁴ Pa.

Preferably, the neutral gas is argon.

The invention even allows the use of argon of commercial quality (generally with 5 ppm of O₂).

The parts to be assembled are subject, for example in the oven, to a thermal vacuum cycle, notably in a secondary vacuum, or in a neutral gas atmosphere.

Thus, the assembly formed by the parts and the brazing material composition may be brought to the brazing temperature observing a preferably “slow” rise in temperature, with one or several ramps of temperature from room temperature.

This rise in temperature may for example be achieved with one or several temperature ramps, each at a rate from 1° C. to 20° C./minute.

The brazing plateau is generally achieved at a temperature, which is the brazing temperature, preferably greater by at least 10° C., and still preferably by at least 30° C., to the melting temperature or liquidus temperature of the brazing material composition, the brazing alloy retained.

Thus, the brazing temperature for the preferred brazing material compositions, brazing alloys A, B, C, D applied according to the invention, may be comprised between a minimum brazing temperature of 980° C. and a maximum brazing temperature of 1,450° C., depending on the brazing material composition.

Indeed, depending on the compositions, the temperature of the liquidus generally varies from 950° C. to 1,420° C. and the brazing temperature will therefore for example vary from 980° C. to 1,450° C. as specified above.

980° C. were mentioned for the lowest point of the brazing temperature since the method even operates with a difference of 15° C. between the melting temperature and the brazing temperature, but a difference of at least 30° C. is generally preferable for large parts, and the lowest brazing temperature is then 1,000° C. Similarly, the highest brazing temperature may be 1,470° C.

Such a melting temperature of the compositions according to another advantage of the method of the invention allows the use of the assembly up to 750° C. for the less refractory alloy D and even up to 1,100° C. for the C alloy and up to 1,200° C. for the A and B alloys.

Table 2 below, indicates the minimum and maximum brazing temperatures for brazing material compositions, the composition of which, expressed in atomic percentages of silicon is located in the preferred composition domains for the brazing compositions, the A, B, C and D brazing alloys.

TABLE 2

| | Preferred | | |
|---|---|---|---|
| Brazing | composition range | Brazing | Brazing |
| alloy | (at. % Si) | Tmin (° C.) | Tmax (° C.) |
| A | 90 to 97 | 1400° C. | 1,450° C. |
| B | 83 to 88 | 1360° C. | 1380° C. |
| | 88 to 97 | 1380° C. | 1,450° C. |
| C | 90 to 97 | 1380° C. | 1,450° C. |
| D | 11 to 20 | 980° C. | 1020° C. |
| | 20 to 30 | 1020° C. | 1200° C. |
| | 30 to 50 | 1200° C. | 1300° C. |
| | 50 to 90 | 1300° C. | 1420° C. |

By applying a brazing temperature which is located in the ranges defined above, good attachment and very good wetting of the pyrocarbon, notably with a contact angle of less than 60°, are achieved, with fast wetting kinetics, i.e. as soon as the total melting of the brazing composition, as shown by the laid drop test conducted with these brazing material compositions. Thus, it is possible (see Example 1) to obtain a contact angle of less than 60° after brazing for a period of time at 1420° C., with a brazing alloy having a composition of 96% of Si, and 4% of Zr in atomic percentages.

This excellent wetting is indispensable in order that the quality of the bond formed between each bead and the substrate is good, since it ensures a good quality of the filling of the joints, but it does not always guarantee good chemical behavior since the latter property is not predictable.

Now, surprisingly, the joints prepared with brazing compositions according to the invention also have excellent mechanical properties.

Mechanical tests (see Examples) carried out on assemblies prepared according to the invention have shown that there was no detachment, no lack of cohesion and during the laid drop test, no detachment or lack of cohesion of the beads upon cooling, and no cracks were observed.

The brazing temperature defined above from 980° C. to 1,450° C., is maintained for a duration of a few seconds (for example 2, 3, 5, 10, 20, 30 seconds) to 60 minutes, preferably from a few seconds (for example 2, 3, 5, 10, 20, 30 seconds) to 20 minutes, still preferably from a few seconds (for example 2, 3, 5, 10, 20, 30 seconds) to 5 minutes notably for brazing beads (case of implants), this is what is called the brazing plateau, or from 10 to 20 minutes for the brazing of larger surfaces to be brazed than in the case of beads for implants.

The duration of the brazing plateau depends on the size of the parts to be assembled and more specifically on the dimensions of the surfaces to be brazed. Indeed, it is possible to attain 30 or even 60 minutes for very large parts with large surface areas raised, i.e. typically at least 50×50 mm².

The specific temperature of the selected brazing plateau depends on the composition of the brazing alloy.

A homogenization plateau for example at 1,100° C. to 1,200° C. for example 1,150° C. for the A, B, C, alloys and at 750° C. for the D alloy, is recommended or even indispensable for large parts (typically from 50×50 mm²) in order to guarantee thermal homogeneity at the parts (substrate and added elements) to be assembled.

It should be noted that as wetting kinetics are already good, it is not necessary to accelerate the wetting, already excellent, this first plateau is therefore in the case of the brazing compositions applied according to the invention, essentially or even exclusively a homogenization plateau.

This plateau may be replaced with a slow rise in temperature, for example at a rate of only 0.5° C./minute, up to 1,100° C. for the A, B, C alloys or 750° C. for the D alloy.

The temperature rise rates mentioned herein depend on the capability of the brazing oven and on the thermal homogeneity inside the oven.

The duration of the first optional plateau as well as the duration of the brazing plateau depends on the size of the oven, on the dimensions of the parts to be brazed and on the tools for holding the parts to be brazed.

This first plateau which is therefore a homogenization plateau is generally observed at a temperature from 1,100° C. to 1,200° C. (for the alloys A, B, C), for example 1,150° C. for a recommended minimum duration of 30 minutes, for example a duration from 30 to 60 minutes, before achieving the brazing plateau, strictly speaking, under the conditions already mentioned above.

Such a first plateau is not indispensable for small parts. Such a first plateau is generally recommended or even indispensable in these two configurations for large parts, i.e. generally parts which have surface areas to be brazed greater than 50×50 mm², in order to guarantee thermal homogeneity at the parts to be assembled.

Or else, thermal homogenization may also be obtained by suppressing this first plateau and by carrying out a slow rise in temperature (at a rate for example of 0.5° C./minute) generally between 1,100° C. and 1,200° C., for the A, B, C alloys for example, around 1,150° C., so that the duration of the assembly exposed to this temperature domain is for example of the order of 30 to 60 minutes.

Like the first plateau, such a slow temperature rise is recommended or even indispensable for large parts.

At the end of the brazing cycle, following the brazing plateau, the assembly is cooled down to room temperature, while observing one or several temperature ramps, at a rate for each of these ramps for example of 5° C. or 6° C. per minute.

During the cooling, the brazing material solidifies and the assembly of the parts in a material based on silicon carbide is effective.

The invention will now be descried with reference to several examples, given as an illustration but not as a limitation.

### EXAMPLES

In these examples, laid drop tests conducted on a zirconia substrate coated with pyrocarbon and assembling tests with the brazing method according to the invention between zirconia discs coated with pyrocarbon and zirconia beads coated with pyrocarbon, are described.

### Example 1

This example describes laid drop tests conducted with a brazing material composition, a brazing alloy according to the invention with a composition of 96% of Si, 4% of Zr in atomic percentages, on a zirconia substrate in the form of a disc with a diameter of 16 mm, coated with a pyrocarbon coating with a thickness of 300 μm.

a) Preparation of the Brazing Material Composition and of the Brazing Slurry, Paste.

The brazing material with a targeted composition of 96 atomic % of Si and 4 atomic % of Zr was prepared from Si powder and ZrSi₂ powder.

These powders were weighed so as to observe the Zr and Si proportions in the composition of the brazing alloy and mixed in a “Turbula”.

An organic binder (cement 650 from NICROBRAZ® or gel Vitta®) was added to this mixture of powders in order to form a viscous slurry, paste.

b) “Laid Drop” Test at 1,420° C.

The thereby prepared brazing slurry, paste is used for forming a small heap of brazing material with a mass to the order of 50 mg. This heap of brazing material is deposited on a disc with a diameter of 16 mm made of zirconia with a pyrocarbon coating with a thickness of 300 μm.

The zirconia plate coated with pyrocarbon was cleaned beforehand with acetone and then with ethanol and finally dried.

The assembly of the heap of brazing material and of the disc is placed in a brazing oven, in the present case, a metal oven, and subjected to a thermal cycle in a secondary vacuum as far as a first 15 minute plateau at 1,340° C. (homogenization without melting the brazing material) and up to a second plateau at 1,420° C. for 1 min.

The heap of brazing material melts during this heat treatment and forms a drop which is called a “laid drop”.

A window gives the possibility of following in situ the spreading out of the drop.

The wetting angle, or contact angle of the drop is measured on the liquid drop in situ through the window.

The wetting angle is less than 60° as soon as the total melting of the brazing material occurs around 1400° C., which corresponds to very good wetting.

After cooling, it is observed that there is no lack of cohesion between the solidified drop and the substrate.

Next, the zirconia substrate coated with pyrocarbon and its solidified brazing material drop were cut out, coated and polished and observed with scanning electron microscopy.

The absence of any lack of cohesion at the interface is shown at the scale of scanning electron microscopy, this qualitative information allows the conclusion to be drawn that the formed bonds are strong.

Let us also note the absence of any crack in the brazing material and the substrate, which is favorable from a mechanical behavioral point of view.

The pyrocarbon/brazing material interface has very limited reactivity at the scale of scanning electron microscopy, reactivity which is associated with a slight dissolution of carbon in the brazing material.

There is no stripping of the substrate.

### Example 2

This example describes the preparation of bonds, assemblies between carbon parts coated with pyrocarbon on the one hand and zirconia beads coated with pyrocarbon on the other hand, by applying the brazing material method according to the invention with a brazing composition, a brazing alloy according to the invention consisting of 96% of Si and 4% of Zr, in atomic percent.

This example further describes the characterization of the assemblies in scanning electron microscopy.

a) Preparation of the Brazing Material Composition, of the Brazing Material Slurry, Paste and of the Parts and Beads to be Assembled.

The targeted brazing material composition i.e. 96% atomic of Si and 4% atomic of Zr, was prepared as described in Example 1.

A brazing material composition slurry, paste was formed like in Example 1.

The zirconia parts coated with pyrocarbon are discs with a diameter of 16 mm and a thickness of 2 mm.

The discs each include a pyrocarbon coating with a thickness of 300 μm right through the disc, in other words on each of the faces of the disc.

The discs coated with pyrocarbon are cleaned with acetone and then with ethanol and finally dried.

Seven discs referenced as no. 1 to no 7 were thus prepared.

These seven discs are then covered with a brazing material slurry, paste on one of their faces which is the face which will receive the beads (with a brazing material amount of 13 mg, 20 mg, 21 mg, 37 mg, 80 mg, 99 mg and 170 mg respectively).

The beads are then deposited on these discs, on the face covered with the brazing slurry, paste with a spatula.

The beads are zirconia beads coated with pyrocarbon.

The grain size of the beads varies between 2 mm and 500 μm.

The pyrocarbon coating has a thickness of the order of 300 μm to 500 μm.

The amounts of beads deposited on the discs no. 1 to no. 7 are 32 mg, 46 mg, 32 mg, 70 mg, 80 mg, 99 mg and 60 mg, respectively.

b) Brazing.

The seven discs and beads assemblies put into contact and ready to be brazed are placed in a brazing oven which is a metal oven in the present case and subjected to a thermal cycle in a secondary vacuum up to 1,420° C., a plateau with a duration of 5 minutes being observed at this temperature.

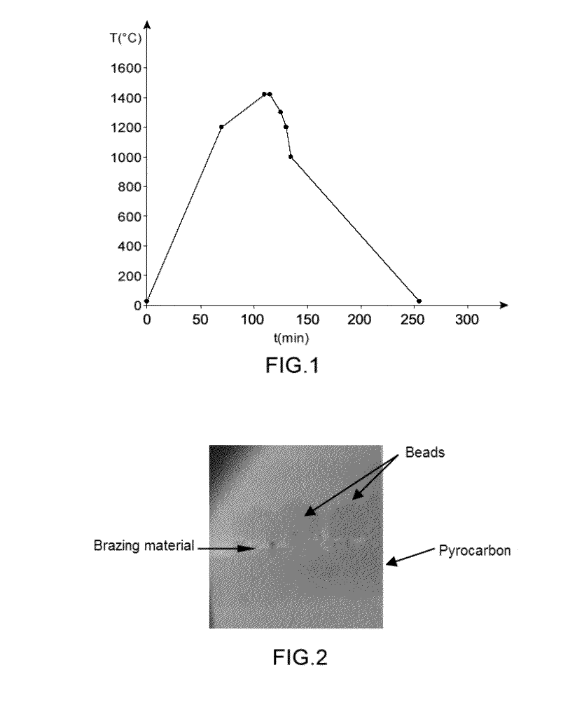

The thermal brazing cycle is illustrated in FIG. 1.

The disc no. 2 is placed in front of the window of the oven in order to follow the brazing in situ.

At the moment of the melting, the observation in situ of the disc no. 2 shows good wetting of the base of the beads with the brazing material, without covering the whole surface of the beads (FIG. 2).

On the photograph of FIG. 2, the brazing material appears bright between the pyrocarbon of the disc and the beads.

c) Observation of the Joint.

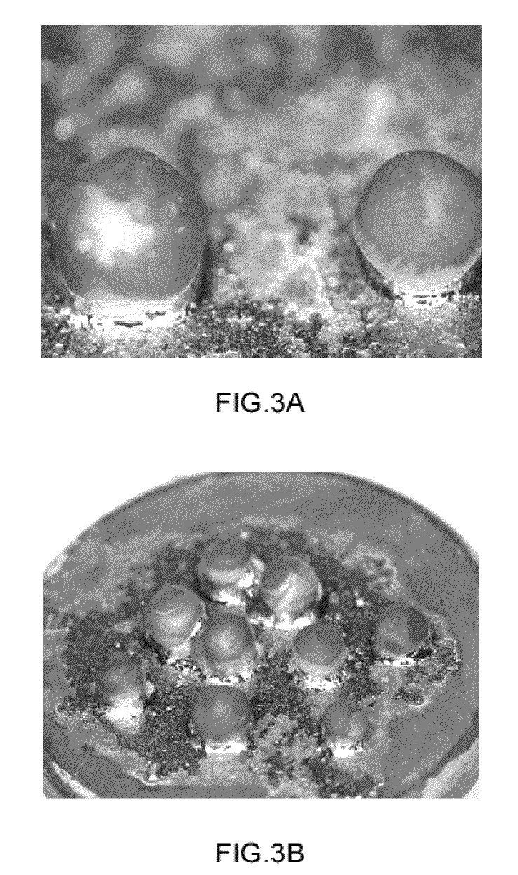

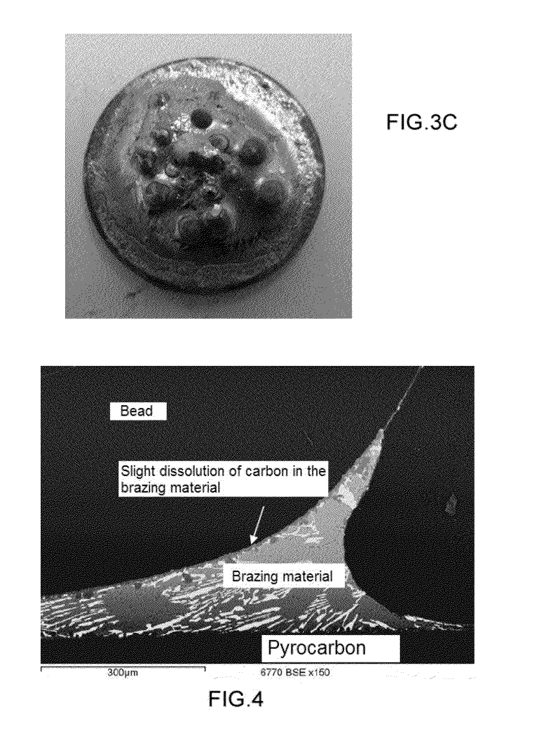

After cooling, the beads are well assembled on their seven discs as demonstrated by the photographs of FIGS. 3A and 3B which show the assembled beads on disc no. 2.

In the case of disc no. 7, the beads are partially embedded into the brazing material as this may be observed in FIG. 3C which shows the assembled beads on disc no. 7.

The joints were characterized by scanning electron microscopy.

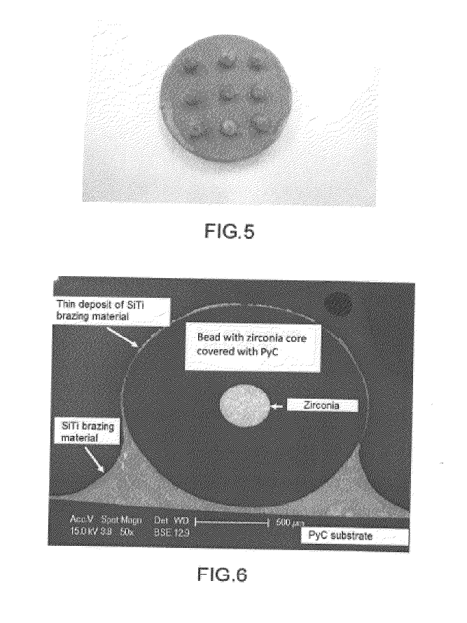

There is no “fault”, “lack”, and very limited reactivity between the pyrocarbon and the brazing material, a reactivity associated with a slight dissolution of the carbon into the brazing material, is shown at the scale of observation in scanning electron microscopy (FIG. 4).

### Example 3

This example describes a laid drop test conducted with a brazing material composition, a brazing alloy according to the invention with a composition of 90.6% of Si, 9.4% of Ti in atomic percentages, on a zirconia substrate with the shape of a disc of a diameter of 16 mm, coated with a pyrocarbon coating of a thickness of 300 μm.

a) Preparation of the Brazing Material Composition and of the Brazing Material Slurry, Paste.

The targeted brazing material composition with 90.6% atomic of Si and 9.4% atomic of Ti, was prepared from Si powder and TiSi₂ powder.

These powders were weighed so as to observe the proportions of Ti and of Si in the brazing composition and mixed in a “Turbula”.

An organic binder (cement 650 from NICROBRAZ®) was added to this mixture of powders in order to form a viscous slurry, paste.

b) “Laid Drop” Test at 1,420° C.

The thereby prepared brazing material slurry, paste is used for forming a small heap of brazing material with a mass of the order of 20 mg. This heap of brazing material is deposited on a disc with a diameter of 16 mm of zirconia coated with a pyrocarbon coating with a thickness of 300 μm.

The zirconia plate coated with pyrocarbon was cleaned beforehand with acetone and then with ethanol and finally dried.

The assembly of the heap of brazing material and disc is placed in a brazing oven, in the present case a metal oven, and subjected to a thermal cycle in a secondary vacuum up to a single plateau of 5 minutes at 1,420° C.

The heap of brazing material melts during the heat treatment and forms a drop which is a called a “laid drop”.

With a window, it is possible to follow the spreading-out of the drop in situ.

The wetting, contact angle of the drop is measured on the liquid drop in situ through the window.

The wetting angle is less than 30° as soon as the brazing material totally melts. This is an excellent wetting.

After cooling, it is observed that there is no lack of cohesion between the solidified drop and the substrate.

### Example 4

This example describes the preparation of bonds, assemblies between carbon parts coated with pyrocarbon on the one hand and zirconia beads coated with pyrocarbon on the other hand, by applying the brazing method according to the invention with a brazing material composition, a brazing alloy according to the invention consisting of 90.6% of Si and 9.4% of Ti in atomic percentages.

This example further describes the characterization of the assembly in scanning electron microscopy.

a) Preparation of the Brazing Material Composition, of the Brazing Material Slurry and of the Parts and Beads to be Assembled.

The targeted brazing material composition i.e. with 90.6 atomic % of Si and 9.4 atomic % of Ti was prepared as described in Example 3.

A brazing material composition slurry was formed like in Example 3.

The zirconia part coated with pyrocarbon is a disc with a diameter of 16 mm and a thickness of 2 mm.

The disc includes a pyrocarbon coating with a thickness of 300 μm right through the disc, in other words on each of the faces of the disc.

This disc coated with pyrocarbon was cleaned with acetone and then with ethanol and finally dried. This disc is also covered with brazing slurry on the face which will receive the beads with a brazing amount of 42 mg.

The beads are then deposited on a disc, on the face coated with brazing material slurry with a spatula.

The beads are in zirconia coated with pyrocarbon.

The grain size of the beads varies between 2 mm and 500 μm.

The pyrocarbon coating has a thickness in the order of 300 μm to 500 μm.

The amount of beads deposited on the disc is 50 mg.

b) Brazing

The disc and beads assembly put into contact and ready to be brazed is placed in a brazing oven which is a metal oven in the present case and subjected to a thermal cycle in a secondary vacuum up to a single plateau at 1,420° C. for 15 minutes.

The disc is placed in front of the window of the oven in order to follow in situ the brazing.

At the moment of melting, the observation in situ of the disc shows good wetting of the base of the beads with the brazing material with slight covering of the surface of the beads.

c) Observation of the Joint.

After cooling, the beads are well assembled on the disc as shown via the photograph of FIG. 5.

The joints were characterized by scanning electron microscopy.

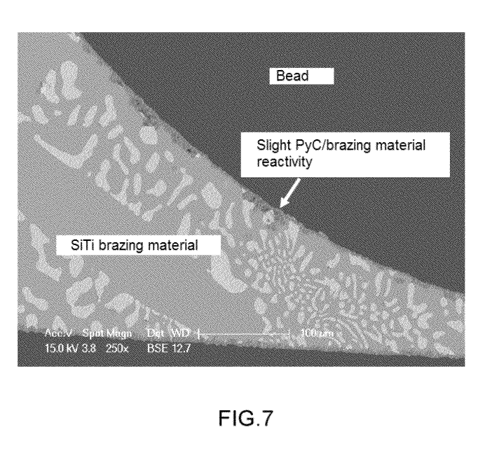

There is no “lack”. Very limited reactivity is observed between the pyrocarbon and the brazing material, a reactivity associated with slight dissolution of carbon into the brazing material. This reactivity is demonstrated at the observation scale of scanning electron microscopy (FIGS. 6 and 7). A slight covering of the beads is observed.

More specifically, in FIG. 6, a bead with a zirconia core covered with pyrocarbon (PyC), the pyrocarbon substrate, the SiTi brazing material between the bead and the substrate, and a thin deposit of SiTi brazing material on the surface of the bead are observed.

In FIG. 7, slight reactivity between the pyrocarbon of the bead and the brazing material and between the pyrocarbon and the substrate and the brazing material is demonstrated.

### Example 5

This example describes the preparation of a bond, an assembly between a carbon part coated with pyrocarbon on the one hand and a zirconia bead coated with pyrocarbon on the other hand, by applying the brazing method according to the invention with a brazing material composition, a brazing alloy according to the invention consisting of 90.6 atomic % of Si and 9.4 atomic % of Ti.

a) Preparation of the Brazing Material Composition, of the Brazing Material Slurry, Paste and of the Parts and Bead to be Assembled.

The targeted brazing material composition, i.e. with 90.6 atomic % of Si and 9.4% atomic of Ti was prepared as described in Example 3.

A brazing material composition slurry, paste was formed like in Example 3.

The zirconia parts coated with pyrocarbon is a disc with a diameter of 16 mm and a thickness of 2 mm.

The disc comprises a pyrocarbon coating with a thickness of 300 μm right through the disc, in other words on each of the faces of the disc.

This disc coated with pyrocarbon was cleaned with acetone and then ethanol and finally dried. This disc is then covered over an area of 5×5 mm² with the brazing material slurry on the face which will receive the bead with respectively a brazing material amount of 2 mg for the single bead.

The bead is then deposited on the disc, on the area coated with the brazing material slurry with a spatula.

The bead is made of zirconia coated with pyrocarbon.

The grain size of the bead is comprised between 2 mm and 500 μm.

The pyrocarbon coating has a thickness of the order of 300 μm to 500 μm.

b) Brazing

The disc and bead assembly put into contact and ready to be brazed is placed in a brazing oven—which is a metal oven in the present case—and subject to a thermal cycle under argon up to a single plateau at 1,320° C. for 5 minutes.

The disc is placed in front of the window of the oven in order to follow the brazing in situ.

Upon melting, observation in situ of the disc shows good wetting of the base of the bead with the brazing material with partial covering of the surface of the bead.

c) Observation of the Bead.

After cooling, the bead is well assembled on the disc. The top of the bead was characterized by scanning electron microscopy. Partial covering of the bead is observed.

### Example 6

This example describes laid drop tests conducted with a brazing material composition, a brazing alloy according to the invention with a composition of 77.5% of Si, 22.5% of Co in atomic percentages, on a zirconia substrate having the shape of a disc with a diameter of 16 mm, coated with a pyrocarbon coating with a thickness of 300 μm.

a) Preparation of the Brazing Material Composition and of the Brazing Slurry.

The targeted brazing material composition with a 77.5 atomic % of Si and 22.5 atomic % of Co, was prepared from a powder of Si and a powder of CoSi₂.

These powders were weighed so as to observe the Co and Si proportions in the brazing material composition and mixed in a “Turbula”.

An organic binder (cement 650 from NICROBRAZ®) was added to this mixture of powders in order to form a viscous slurry.

b) “Laid Drop” Test at 1,320° C.

The thereby prepared brazing material slurry is used for forming a small heap of brazing material with a mass of the order of 50 mg. This heap of brazing material is deposited on a zirconia disc with a diameter of 16 mm coated with pyrocarbon.

The zirconia plate coated with pyrocarbon was cleaned beforehand with acetone and then ethanol and finally dried.

The assembly of the heap of brazing material and of the disc is placed in a brazing oven, in the present case, a metal oven and subjected to a thermal cycle in a secondary vacuum up to a single 5 minute plateau at 1,320° C.

The heap of brazing material melts during this heat treatment and forms a drop which is called a “laid drop”.

A window gives the possibility of following in situ the spreading-out of the drop.

The wetting, contact angle of the drop is measured on the liquid drop in situ through the window.

The wetting angle is less than 60° as soon as the brazing totally melts, i.e. it is a good wetting.

After cooling, it is observed that there is no lack of cohesion between the solidified drop and the substrate.

### Example 7

This example describes the preparation of bonds, assemblies between carbon parts coated with pyrocarbon on the one hand and zirconia beads coated with pyrocarbon on the other hand, by applying the brazing method according to the invention with a brazing material composition, a brazing alloy according to the invention consisting of 77.5% of Si and 22.5% of Co, in atomic percentages.

This example further describes the characterization of the assemblies by scanning electron microscopy.

a) Preparation of the Brazing Material Composition, of the Brazing Material Slurry and of the Parts and Beads to be Assembled.

The targeted brazing material composition, i.e. 77.5 atomic % of Si and 22.5 atomic % of Co, was prepared as described in Example 6.

A brazing material composition slurry was formed like in Example 6.

The zirconia parts coated with pyrocarbon are discs with a diameter of 16 mm and a thickness of 2 mm.

The discs each include a pyrocarbon coating with a thickness of 300 μm on either side of the disc, in other words on each of the faces of the disc.

The disc coated with pyrocarbon was cleaned with acetone then with ethanol and finally dried.

This disc was then covered with a brazing paste on the face which will receive the beads with a brazing material amount of 29 mg.

The beads are then deposited on these discs, on the face coated with the brazing slurry with a spatula.

The beads are made of zirconia coated with pyrocarbon.

The grain size of the beads varies between 2 mm and 500 μm.

The pyrocarbon coating has a thickness to the order of 300 μm to 500 μm.

The amount of beads deposited on the disc is 45 mg.

b) Brazing.

The disc and beads assembly put into contact and ready to be brazed is placed in a brazing oven—which is a metal oven in the present case—and subject to a thermal cycle under argon up to 1270° C. for 30 seconds.

The disc is placed in front of the window of the oven in order to follow the brazing in situ.

Upon melting, observation in situ of the disc shows good wetting of the base of the beads with the brazing material, without covering the whole surface of the beads.

c) Observation of the Joint.

After cooling, the beads are well assembled on the disc.

The joints were characterized by scanning electron microscopy.

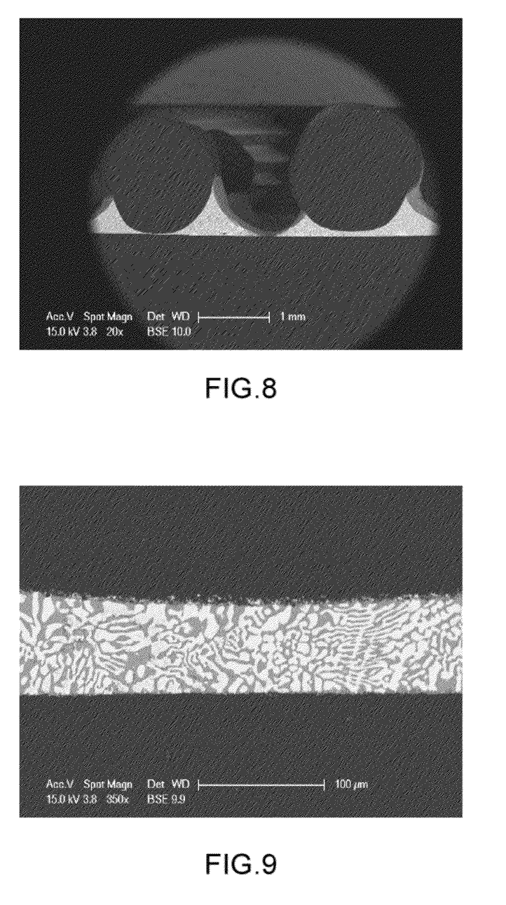

There is no “lack” and very limited reactivity between the pyrocarbon and the brazing material, a reactivity associated with slight dissolution of the carbon in the brazing material, is shown at the scale of observation in scanning electron microscopy (FIGS. 8 to 10).

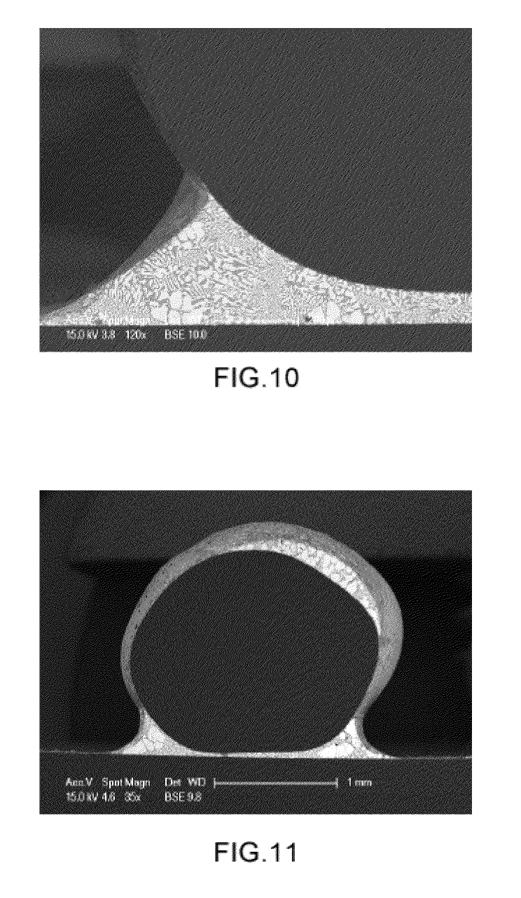

More specifically, FIG. 8 shows the assembly between two beads and the substrate and FIGS. 9 and 10 show the joint between a bead and the substrate.

### Example 8

This example describes the preparation of the bonds, assemblies between carbon parts coated with pyrocarbon on the one hand and zirconia beads coated with pyrocarbon on the other hand, by applying the brazing method according to the invention with a brazing material composition, a brazing alloy according to the invention consisting of 77.5% of Si and of 22.5% of Co, in atomic percentages.

This example further describes the characterization of the assembly in scanning electron microscopy.

a) Preparation of the Brazing Material Composition, of the Brazing Material Slurry and of the Parts and Beads to be Assembled.

The brazing material having the targeted composition i.e. 77.5 atomic % of Si, and 22.5 atomic % of Co was prepared as described in Example 6.

A brazing material composition slurry was formed like in Example 6.

The zirconia parts coated with pyrocarbon are discs with a diameter of 16 mm and a thickness of 2 mm.

The discs each include a pyrocarbon coating with a thickness of 300 μm on either side of the disc, in other words on each of the faces of the disc.

The disc coated with pyrocarbon was cleaned with acetone and then with ethanol and finally dried.

This disc was then covered with a brazing material slurry on the face which will receive the beads with a brazing material amount of 29 mg.

The beads were then deposited on the disc, on the face coated with brazing slurry, with a spatula.

The beads are made of zirconia coated with pyrocarbon.

The grain size of the beads varies between 2 mm and 500 μm.

The pyrocarbon coating has a thickness of the order of 300 μm to 500 μm.

The amount of beads deposited on the disc is 45 mg.

b) Brazing.

The disc and beads assembly put into contact and ready to be brazed is placed in a brazing oven—which is a metal oven in the present case—and subjected to a thermal cycle under argon up to 1,320° C. for 5 minutes.

The disc is placed in front of the window of the oven in order to follow the brazing in situ.

Upon melting, the observation in situ of the disc shows good wetting of the beads with the brazing material with covering of the whole surface of the beads.

c) Observation of the Joint.

After cooling, the beads are well assembled on the disc.

The beads and assemblies with the substrate were characterized by scanning electron microscopy.

There is no “lack” and very limited reactivity between the pyrocarbon and the brazing material, said reactivity being associated with slight dissolution of the carbon in the brazing material, is shown at the scale of observation in scanning electron microscopy (FIGS. 11 and 12).

More specifically, FIG. 11 shows the assembly between a bead and the substrate, and FIG. 12 shows the interface between pyrocarbon and the brazing material and demonstrates slight reactivity (reaction area in grey in FIG. 12).

### Example 9

Cytotoxicity, biocompatibility tests were conducted on pyrocarbon samples coated with Zr—Si, Co—Si, and Ti—Si brazing materials.

These tests were conducted according to the ISO 10993 standard with cultures of mouse fibroblast cells, L-929.

No cell destruction and no toxicity of the samples on the cells was shown during these tests.

### REFERENCES

[1] FR-A1-2 893 247
[2] EP-A1-1 112 753
[3] WO-A1-01/32950
[4] WO-A1-01/68557
[5] WO-A1-01/68560

1-19. (canceled)

20. A method for assembling a substrate comprising at least one external pyrocarbon surface with parts each comprising at least one external pyrocarbon surface, comprising:
depositing a brazing material composition consisting of an alloy of one or several metal(s) or metalloid(s) on at least one portion to be assembled of the external pyrocarbon surface of the substrate;
depositing the parts on the brazing material composition such that the brazing material composition, upon melting, is in contact with the portion to be assembled of the external surface of the substrate, and with a portion to be assembled of the external pyrocarbon surface of each of the parts, wherein the shape of the parts is not modified and remains apparent, so as to leave free, empty spaces between the parts, and so that the parts are not embedded in the brazing material composition;
heating the assembly formed by the substrate, the brazing material composition and the deposited parts in vacuo or in a neutral gas atmosphere at a brazing temperature sufficient for totally or at least partially melting the brazing material composition and less than 1,700° C.;
cooling the substrate, the brazing material composition and the parts until solidification of the brazing material composition.

21. The method according to claim 20, wherein two or more from among the deposited parts are in contact with each other or else all the deposited parts are not in contact with each other.

22. The method according to claim 20, wherein the brazing is carried out at a brazing temperature greater by at least 15° C. than the melting temperature of the brazing material composition.

23. The method according to claim 20, wherein the brazing temperature is from 980° C. to 1,450° C.

24. The method according to claim 20, wherein said brazing material composition is selected from alloys comprising more than 40 atomic % of an element X selected from Ni, Fe, Co, Pd or Pt.

25. The method according to claim 20, wherein the said brazing material composition is selected from alloys based on Ag or on Ag and Cu containing at least one reactive element at a concentration of less than 10 atomic %; and alloys based on Ag containing Si at a concentration of less than 40 atomic %.

26. The method according to claim 20, wherein said brazing material composition is selected from alloys based on silicon containing Si at a concentration of more than 40 atomic %, and having limited and controlled reactivity with pyrocarbon.

27. The method according to claim 26, wherein said brazing material composition is selected from binary alloys consisting of silicon at a concentration of more than 40 atomic % and of an element selected from the group consisting of Cr, Re, V, Ru, Ir, Rh, Pd, Co, Pt, Ce, Zr, Ti, Ag, Au, Cu, Nd, Pr, Y and Hf.

28. The method according to claim 27, wherein said brazing material composition is selected from the group consisting of binary alloys (A) consisting of silicon and zirconium; binary alloys (B) consisting of silicon and titanium; binary alloys in (C) consisting of silicon and cobalt; and binary alloys (D) consisting of silicon and silver.

29. The method according to claim 28, wherein said brazing composition is a binary alloy (A) consisting of 75% to 97% silicon and of 25% to 3% Zr, in atomic percentages.

30. The method according to claim 28, wherein said brazing composition is a binary alloy (B) consisting of 60% to 97% silicon and from 40% to 3% Ti in atomic percentages.

31. The method according to claim 28, wherein said brazing material composition is a binary alloy (C) consisting of 60% to 97% silicon and of 40% to 3% Co, in atomic percentages.

32. The method according to claim 28, wherein said brazing material composition is a binary alloy (D) consisting of 5% to 97% silicon and of 95% to 3% Ag, in atomic percentages.

33. The method according to claim 20, wherein the substrate consists of pyrocarbon; or the substrate consists of a core made of a first material selected from biocompatible materials selected from zirconia, graphite or silicon carbide; at least one portion of the external surface of the core made of a first material and the totality of the external surface of the core made of a first material being coated with a pyrocarbon coating or skin.

34. The method according to claim 20, wherein the parts consist of pyrocarbon; or each of the parts consist of a core made of a first material selected from biocompatible materials selected from zirconia, graphite or silicon carbide; at least one portion of the external surface of the core made of a first material and the totality of the external surface of the core made of a first material being coated with a pyrocarbon coating or skin.

35. The method according to claim 20, wherein the parts appear as spherical or spheroidal beads, with a grain size comprised between 2.5 mm and 100 micrometers.

36. The method according to claim 20, wherein a brazing material composition powder is formed, said powder is suspended in an organic binder so as to obtain a brazing material composition suspension, paste or slurry, and said suspension, paste or slurry, is deposited on at least one portion to be assembled of the external pyrocarbon surface of the substrate.

37. The method according to claim 20, wherein said substrate is an implant or part of an implant, for prostheses of bone portions; or an interposition implant between bone surfaces.

38. The method according to claim 20, wherein the brazing is carried out at a brazing temperature greater by at least 30° C. than the melting temperature of the brazing material composition.

39. The assembly obtained by the method according to claim 20.
